(19) United States
(12) Patent Application Publication
(10) Pub. No.: US 2012/0004188 A1
(43) Pub. Date: Jan. 5, 2012

(54) USE OF RANOLAZINE FOR TREATING PULMONARY HYPERTENSION

(75) Inventor: Luiz Belardinelli, Palo Alto, CA (US)

(73) Assignee: Gilead Sciences, Inc.

(21) Appl. No.: 13/160,393

(22) Filed: Jun. 14, 2011

Related U.S. Application Data
(60) Provisional application No. 61/355,462, filed on Jun. 16, 2010.
(60) Provisional application No. 61/407,864, filed on Oct. 28, 2010.

(51) Int. Cl. A61K 31/495; A61P 9/12; A61K 31/7048

(52) U.S. Cl. 514/26; 514/252.12

(57) ABSTRACT

This disclosure relates generally to treating patients having pulmonary hypertension such as pulmonary arterial hypertension (PAH), or symptoms associated therewith, by administering a therapeutically effective amount of ranolazine or a salt or salts thereof to the patient.

### CROSS REFERENCE TO RELATED APPLICATIONS

This application claims the benefit under 35 U.S.C. §119(e) of U.S. Provisional Application Ser. No. 61/355,462 filed Jun. 16, 2010 and U.S. Provisional Application Ser. No. 61/407,864, filed Oct. 28, 2010, both of which are incorporated by reference in their entirety.

### FIELD OF THE INVENTION

The invention is directed to methods of treating pulmonary hypertension in patients in need thereof by administering a therapeutically effective amount of ranolazine or a salt or salts thereof.

### STATE OF THE ART

Pulmonary hypertension (PH) has been previously classified as primary (idiopathic) or secondary. Recently, the World Health Organization (WHO) has classified pulmonary hypertension into five groups:

Group 1: pulmonary arterial hypertension (PAH);

Group 2: PH with left heart disease;

Group 3: PH with lung disease and/or hypoxemia;

Group 4: PH due to chronic thrombotic and/or embolic disease; and

Group 5: miscellaneous conditions (e.g., sarcoidosis, histiocytosis X, lymphangiomatosis and compression of pulmonary vessels).

See, for example, Rubin (2004) Chest 126:7-10.

Pulmonary arterial hypertension (PAH) is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. Patients with PAH typically develop significant increases in pulmonary vascular resistance (PVR) and sustained elevations in pulmonary artery pressure (PAP). These pressures make it difficult for the heart to pump blood through the lungs to be oxygenated. Patients with PAH suffer from extreme shortness of breath as the heart struggles to pump against these high pressures, which ultimately lead to right ventricular failure and death. It has been contemplated that dysfunctional right ventricle can lead to a dysfunctional left ventricle. See, Braunwald, “Heart Disease: A Textbook of Cardiovascular Medicine” 1883-1914 (2008). Patients diagnosed with PAH have poor prognosis and, equally, a compromised quality of life, with a mean life expectancy of 2 to 5 years from the time of diagnosis if untreated. The most common cause of death in patients is progressive right-sided heart failure. Id.

Today, medical therapies for treating PAH include digoxin, diuretics, anticoagulants, and supplemental oxygen. Pulmonary vasodilators such as prostacyclins, endothelin receptor antagonists, and phosphodiesterase inhibitors improve exercise capacity in PAH and may indirectly improve right ventricular function via afterload reduction but they do not directly improve RV function or lessen RV ischemia. Galie, N. et al., Eur Heart J, 2009; 30(20):2493-537. Based on small studies of PAH, digoxin may be beneficial as an RV inotrope, but it may increase myocardial oxygen demand. Currently, there are no treatments approved for PAH which directly improve RV function without increasing myocardial oxygen demand. Rich S., et al., Chest 1998; 14(3):787-92. New therapies are needed to approach treatment of PAH via alternate mechanisms.

### SUMMARY

This disclosure is directed to the surprising and unexpected discovery that a patient suffering from pulmonary hypertension may be treated, or have their symptoms be treated, by ranolazine. In a rodent model of chronic pulmonary arterial hypertension (PAH) and right ventricular (RV) dysfunction induced by Monocrotaline (MCT), ranolazine is shown to prevent PAH and RV dysfunction and to reduce pulmonary vascular remodeling. Moreover, the effect of ranolazine in preventing right ventricular remodeling is demonstrated using a murine model of large anterior wall acute myocardial infarction (AMI) produced by permanent coronary artery ligation of the left coronary artery. It is therefore further contemplated that ranolazine improves the right ventricle function of the patient thereby alleviating symptoms of PAH, including exertional dyspnea, fatigue, and chest pain without increasing myocardial oxygen demand.

As such, in one aspect, the disclosure provides a method of treating pulmonary hypertension in a patient in need thereof, said method comprising administering to the patient a therapeutically effective amount of ranolazine or a salt or salts thereof. The pulmonary hypertension, in one aspect, is pulmonary arterial hypertension (PAH) which may be selected from idiopathic PAH, familial PAH, pulmonary veno-occlusive disease (PVOD), pulmonary capillary hemangiomatosis (PCH), persistent pulmonary hypertension of the newborn, or PAH associated with another disease or condition.

In another aspect of the disclosure, provided is a method for improving right ventricle (RV) function in a patient suffering from pulmonary hypertension, comprising administering to the patient a therapeutically effective amount of ranolazine or a salt or salts thereof.

In yet another aspect of the disclosure, provided is a method for reducing pulmonary arterial pressure in a patient in need thereof, comprising administering to the patient a therapeutically effective amount of ranolazine or a salt or salts thereof.

In yet another aspect of the disclosure, provided is a method for treating or ameliorating one or more symptoms in a patient suffering from pulmonary hypertension, comprising administering to the patient a therapeutically effective amount of ranolazine or a salt or salts thereof. In one aspect, the symptoms comprise fatigue. In another aspect, the symptoms comprise exertional dyspnea. In yet another aspect, the symptoms comprise chest pain.

Yet in another aspect, the disclosure provides a method of treating or preventing asymptomatic pulmonary hypertension in a patient in need thereof, said method comprising administering to the patient a therapeutically amount of ranolazine or a salt or salts thereof.

Yet another aspect provides a method of treating pulmonary arterial hypertension (PAH) in a patient in need thereof, said method comprising orally administering to the patient a therapeutically amount of ranolazine or a salt or salts thereof, wherein the therapeutically amount contains an aggregate daily dose of ranolazine in the amount of 75 milligrams, 500 milligrams or 375 milligrams.

### BRIEF DESCRIPTION OF THE DRAWINGS

FIG. 1 are representative images of α-SMA stained lung sections taken from control animals (control), Monocrotaline (MCT) treated animals (MCT) and animals in the high dose (0.5%) ranolazine (RAN) group (MCT+RAN (0.5%) at day 28 following MCT administration.

FIGS. 2A and 2B are charts summarizing digital quantification of the α-SMA staining performed for the entire lung sections. 2A: lumen area arteries >50 μm and 2B: lumen area arteries <50 μm. These charts demonstrate that RAN significantly reduced MCT-induced remodeling in intra-acinar arteries.

FIG. 3 shows that treatment with ranolazine led to a significant preservation of right ventrical (RV) function (measured as tricuspidal annulus plane systolic excursion (TAPSE) and RV fractional area change, right panel) and dimension (measured as RV diastolic and systolic areas, left panel) in comparison to vehicle-treated mice.

### DETAILED DESCRIPTION

Prior to describing this disclosure in greater detail, the following terms will first be defined.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “an additional therapeutic agent” includes a plurality of therapeutic agents.

### 1. Definitions

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

As stated above, the disclosure is directed to a method of treating pulmonary arterial hypertension or pulmonary arterial hypertension (PAH) comprising administering to a patient in need thereof a therapeutically effective amount of ranolazine or a salt or salts thereof.

The term “treatment” means any treatment of a disease in a patient including: (i) preventing the disease, that is causing the clinical symptoms not to develop; (ii) inhibiting the disease progression, that is, arresting the development of clinical symptoms; and/or (iii) relieving the disease, that is, causing the regression of clinical symptoms. By way of example only, treating may include improving right ventricular function and/or alleviating or ameliorating symptoms, including, but not limited to exertional dyspnea, fatigue, and combinations thereof.

As used herein, the term “pulmonary arterial hypertension” or “PAH” is intended to include idiopathic PAH, familial PAH, pulmonary veno-occlusive disease (PVOD), pulmonary capillary hemangiomatosis (PCH), persistent pulmonary hypertension of the newborn, or PAH associated with another disease or condition, such as, but not limited to, collagen vascular disease, congenital systemic-to-pulmonary shunts (including Eisenmenger's syndrome), portal hypertension, HIV infection, drugs and toxins, thyroid disorders, glycogen storage disease, Gaucher disease, hereditary hemorrhagic telangiectasia, hemoglobinopathies, myeloproliferative disorders, or splenectomy.

The term “patient” typically refers to a mammal, such as, for example, a human.

The term “therapeutically effective amount” refers to that amount of a compound, such as ranolazine, that is sufficient to effect treatment, as defined above, when administered to a patient in need of such treatment. The therapeutically effective amount will vary depending upon the specific activity or delivery route of the agent being used, the severity of the patient's disease state, and the age, physical condition, existence of other disease states, and nutritional status of the patient. Additionally, other medication the patient may be receiving will effect the determination of the therapeutically effective amount of the therapeutic agent to administer.

The term “ranolazine” or “RAN” refers to the compound named “±-N-(2,6-dimethylphenyl)-4-[2-hydroxy-3-(2-methoxyphenoxy)-propyl]-1-piperazineacetamide,” and its pharmaceutically acceptable salts. Ranolazine is disclosed in U.S. Pat. No. 4,567,264 for use in the treatment of cardiovascular diseases, including arrhythmias, variant and exercise-induced angina, and myocardial infarction. In its dihydrochloride salt form, ranolazine is represented by the chemical formula:

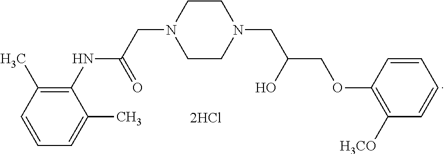

As used herein, the term “salt” or “pharmaceutically acceptable salt” refers to a salt of a compound that is derived from a variety of physiologically acceptable organic and inorganic counter ions. Such counter ions are well known in the art and include, by way of example only, sodium, potassium, calcium, magnesium, aluminum, lithium and ammonium, for example tetraalkylammonium, and the like when the molecule contains an acidic functionality; and when the molecule contains a basic functionality, salts of organic or inorganic acids, such as hydrochloride, sulfate, phosphate, diphosphate, nitrate hydrobromide, tartrate, mesylate, acetate, malate, maleate, fumarate, tartrate, succinate, citrate, lactate, pamoate, salicylate, stearate, methanesulfonate, p-toluenesulfonate, and oxalate, and the like. Suitable pharmaceutically acceptable salts also include those listed in Remington's Pharmaceutical Sciences, 17th Edition, pg. 1418 (1985) and P. Heinrich Stahl, Camille G. Wermuth (Eds.), Handbook of Pharmaceutical Salts Properties, Selection, and Use; 2002. Examples of acid addition salts include those formed from acids such as hydroiodic, phosphoric, metaphosphoric, nitric and sulfuric acids, and with organic acids, such as alginic, ascorbic, anthranilic, benzoic, camphorsulfuric, citric, embonic (pamoic), ethanesulfonic, formic, fumaric, furoic, galacturonic, gentisic, gluconic, glucuronic, glutamic, glycolic, isonicotinic, isothionic, lactic, malic, mandelic, methanesulfonic, mucic, pantothenic, phenylacetic, propionic, saccharic, salicylic, stearic, succinic, sulfinilic, trifluoroacetic and arylsulfonic for example benzenesulfonic and p-toluenesulfonic acids. Examples of base addition salts formed with alkali metals and alkaline earth metals and organic bases include chloroprocaine, choline, N,N-dibenzylethylenediamine, diethanolamine, ethylenediamine, lysine, meglumaine (N-methylglucamine), and procaine, as well as internally formed salts. Salts having a non-physiologically acceptable anion or cation are within the scope of the invention as useful intermediates for the preparation of physiologically acceptable salts and/or for use in non-therapeutic, for example, in vitro, situations.

### 2. Methods

As stated above, the present disclosure relates to methods of treating pulmonary hypertension such as pulmonary arterial hypertension (PAH). The method comprises administering to a patient in need thereof a therapeutically amount of ranolazine or a salt or salts thereof.

In patients with significant pulmonary hypertension, such as PAH, chest pain, exertional dyspnea, and fatigue are common symptoms and most are attributed to right ventricle (RV) ischemia as PAH progresses to RV failure. See, Rich, et al., Ann Intern Med, 1987; 107(2):216-23; Barst, Am. J. Med., 2004; 116(6):427-8. However, pulmonary hypertension may be treated by the methods of the disclosure even if the patient is asymptomatic, e.g., without chest pain, exertional dyspnea, and/or fatigue. In one particular aspect, the patient does not suffer from pain or chest pain.

Ranolazine is an approved medication for the treatment of chronic stable angina in patients with coronary artery disease. Although the exact mechanism underlying the antiischemic/antianginal effect of ranolazine is unknown, recent evidence suggests that ranolazine reduces calcium overload of ischemic and failing myocytes through inhibition of the late sodium current (I_{Na}). Stone, P., Cardiol Clin 2008; 26(4):603-14. It is contemplated that ranolazine can ameliorate calcium overloaded RV myocytes which cause RV diastolic dysfunction. Without being limited to any theory, it is contemplated that by reducing RV diastolic tension, ranolazine improves myocardial blood flow during diastole in the ischemic RV, thereby relieving chest pain, while simultaneously improving RV performance and contractility. This latter effect may increase stroke volume and cardiac output which could translate into benefits in exercise capacity. It is further contemplated that ranolazine will alleviate additional symptoms of PAH including exertional dyspnea and fatigue by improving RV performance and function. For example, as demonstrated by Example 2, ranolazine is shown to reduce pulmonary arterial pressure.

It is demonstrated herein that, in a rodent model of chronic PAH and right ventricular (RV) dysfunction induced by Monocrotaline (MCT), ranolazine prevented PAH and RV dysfunction. Moreover, using a murine model of large anterior wall acute myocardial infarction (AMI) produced by permanent coronary artery ligation of the left coronary artery, the effect of ranolazine in preventing right ventricular remodeling is demonstrated. These data indicate that ranolazine improves the right ventricle function of the patient thereby alleviating symptoms of pulmonary hypertension.

Methods of the present disclosure can be used to reduce pulmonary arterial pressure in patients in need thereof. Normally, the RV differs from the left ventricle (LV) in its pattern of coronary blood flow during the cardiac cycle. Whereas the left coronary artery blood flow occurs primarily in diastole due to the lack of a pressure gradient between the aorta and the left ventricle in systole, right coronary blood flow occurs throughout the cardiac cycle because aortic pressure is much higher than RV pressure during systole and diastole, thereby driving coronary blood flow continuously. In patients with PAH, however, as RV systolic pressure rises and begins to match aortic systolic pressure, there is no longer a gradient between the aorta and RV. Therefore, the RV, which is normally perfused throughout the cardiac cycle, only receives coronary blood flow in diastole. Thus, in patients with advanced PAH, the RV becomes progressively ischemic as pulmonary artery pressures (and RV pressures) rise, which results in further deterioration of an already vulnerable RV. Elevation of RV end-diastolic pressure also contributes to RV ischemia in these patients by impeding coronary blood flow in diastole. Barst, Am. J. Med., 2004; 116(6):427-8; van Wolferen, et al., Eur Heart J 2008; 29(1): 120-7.

### Pulmonary Hypertension, Classification and Clinical Parameters

The pulmonary hypertension condition treated by the methods of the disclosure can comprise any one or more of the conditions recognized according to the World Health Organization (WHO) or Venice (2003) classification (see, for example, Rubin (2004) Chest 126:7-10):

Group 1: Pulmonary arterial hypertension (PAH)
1.1 idiopathic PAH
1.2 familial PAH
1.3 PAH associated with:
   1.3.1 collagen vascular disease
   1.3.2 congenital systemic-to-pulmonary shunts (including Eisenmenger's syndrome)
   1.3.3 portal hypertension
   1.3.4 HIV infection
   1.3.5 drugs and toxins
   1.3.6 other (thyroid disorders, glycogen storage disease, Gaucher disease, hereditary hemorrhagic telangiectasia, hemoglobinopathies, myeloproliferative disorders, splenectomy)
1.4 PAH associated with significant venous or capillary involvement
   1.4.1 pulmonary veno-occlusive disease (PVOD)
   1.4.2 pulmonary capillary hemangiomatosis (PCH)
1.5 persistent pulmonary hypertension of the newborn

Group 2: Pulmonary hypertension with left heart disease
2.1 left-sided atrial or ventricular heart disease
2.2 left-sided valvular heart disease

Group 3: Pulmonary hypertension associated with lung diseases and/or hypoxemia
3.1 chronic obstructive pulmonary disease (COPD)
3.2 interstitial lung disease
3.3 sleep-disordered breathing
3.4 alveolar hypoventilation disorders
3.5 chronic exposure to high altitude
3.6 developmental abnormalities

Group 4: Pulmonary hypertension due to chronic thrombotic and/or embolic disease
4.1 thromboembolic obstruction of proximal pulmonary arteries
4.2 thromboembolic obstruction of distal pulmonary arteries
4.3 non-thrombotic pulmonary embolism (tumor, parasites, foreign material)

Group 5: Miscellaneous (sarcoidosis, histiocytosis X, lymphangiomatosis, compression of pulmonary vessels (adenopathy, tumor, fibrosing mediastinitis))

In one aspect, the pulmonary hypertension condition comprises PAH (WHO Group 1), for example idiopathic PAH, familial PAH or PAH associated with another disease or condition.

Pulmonary hypertension at baseline can be mild, moderate or severe, as measured for example by WHO functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system:

Class I: pulmonary hypertension without resulting limitation of physical activity; ordinary physical activity does not cause undue dyspnea or fatigue, chest pain or near syncope;

Class II: pulmonary hypertension resulting in slight limitation of physical activity; patient comfortable at rest; ordinary physical activity causes undue dyspnea or fatigue, chest pain or near syncope;

Class III: pulmonary hypertension resulting in marked limitation of physical activity; patient comfortable at rest; less than ordinary activity causes undue dyspnea or fatigue, chest pain or near syncope;

Class IV: pulmonary hypertension resulting in inability to carry out any physical activity without symptoms; patient manifests signs of right-heart failure; dyspnea and/or fatigue may be present even at rest; discomfort is increased by any physical activity.

In one aspect, the methods are directed to treating Class I, also known as asymptomatic pulmonary hypertension.

In one aspect, the subject at baseline exhibits pulmonary hypertension (e.g., PAH) of at least WHO Class II, for example WHO Class II or Class III.

In another aspect, the subject at baseline exhibits mean PAP at rest of at least about 30 mmHg, for example at least about 35, at least about 40, at least about 45 or at least about 50 mmHg.

The methods of the present disclosure, when applied to a subject, can achieve one or more of the following objectives:

(a) adjustment of one or more hemodynamic parameters towards a more normal level, for example lowering mean PAP or PVR, or raising Pulmonary Capillary Wedge Pressure (PCWP) or Left Ventricular End-Diastolic Pressure (LVEDP), versus baseline;

(b) improvement of pulmonary function versus baseline, for example increasing exercise capacity, illustratively as measured in a test of 6-minute walking distance (6MWD), or lowering Borg dyspnea index (BDI);

(c) improvement of one or more quality of life parameters versus baseline, for example an increase in score on at least one of the SF-36® health survey functional scales;

(d) general improvement versus baseline in the severity of the condition, for example by movement to a lower WHO functional class;

(e) improvement of clinical outcome following a period of treatment, versus expectation in absence of treatment (e.g., in a clinical trial setting, as measured by comparison with placebo), including improved prognosis, extending time to or lowering probability of clinical worsening, extending quality of life (e.g., delaying progression to a higher WHO functional class or slowing decline in one or more quality of life parameters such as SF-36® health survey parameters), and/or increasing longevity; and/or

(f) adjustment towards a more normal level of one or more molecular markers that can be predictive of clinical outcome (e.g., plasma concentrations of endothelin-1 (ET-1), cardiac troponin T (cTnT) or B-type natriuretic peptide (BNP)).

What constitutes a therapeutically effective amount of ranolazine for treating pulmonary hypertension, or in particular, PAH, can vary depending on the particular pulmonary hypertension condition to be treated, the severity of the condition, body weight and other parameters of the individual subject, and can be readily established without undue experimentation by the physician or clinician based on the disclosure herein. However, contemplated doses are described below.

Various clinical parameters and standards to measure the effectiveness of a pulmonary hypertension therapy are described below and are known in the art as well. Accordingly, the effectiveness of ranolazine can be measured by these parameters or standards. Additionally, the relative effectiveness of ranolazine, as compared to other agents, can be determined with these clinical parameters or standards, as well as in a non-clinical setting. Examples of such non-clinical settings include, without limitation, an animal model. Non-limiting examples of animal models are provided in Examples.

### A. Improvement on Clinical Parameters

In one aspect, the subject being treated experiences, during or following the treatment period, at least one of

(a) adjustment of one or more hemodynamic parameters indicative of the pulmonary hypertension condition towards a more normal level versus baseline;

(b) increase in exercise capacity versus baseline;

(c) lowering of Borg Dyspnea Index (BDI) versus baseline;

(d) improvement of one or more quality of life parameters versus baseline; and/or

(e) movement to a lower WHO functional class.

Any suitable measure of exercise capacity can be used; a particularly suitable measure is obtained in a 6-minute walk test (6MWT), which measures how far the subject can walk in 6 minutes, i.e., the 6-minute walk distance (6MWD).

The Borg dyspnea index (BDI) is a numerical scale for assessing perceived dyspnea (breathing discomfort). It measures the degree of breathlessness after completion of the 6 minute walk test (6MWT), where a BDI of 0 indicates no breathlessness and 10 indicates maximum breathlessness.

In various aspects, an effective amount of a pulmonary hypertension therapy adjusts one or more hemodynamic parameters indicative of the pulmonary hypertension condition towards a more normal level. In one such aspect, mean PAP is lowered, for example by at least about 3 mmHg, or at least about 5 mmHg, versus baseline. In another such aspect, PVR is lowered. In yet another such aspect, PCWP or LVEDP is raised.

In various aspects, an effective amount of a pulmonary hypertension therapy improves pulmonary function versus baseline. Any measure of pulmonary function can be used; illustratively 6MWD is increased or BDI is lowered.

In one such aspect, 6MWD is increased from baseline by at least about 10 meters, for example at least about 20 meters or at least about 30 meters. In many instances, the method of the present embodiment will be found effective to increase 6MWD by as much as 50 meters or even more.

In another such aspect, BDI, illustratively as measured following a 6MWT, is lowered from baseline by at least about 0.5 index points. In many instances, the method of the present embodiment will be found effective to lower BDI by as much as 1 full index point or even more.

The SF-36® health survey provides a self-reporting, multi-item scale measuring eight health parameters: physical functioning, role limitations due to physical health problems, bodily pain, general health, vitality (energy and fatigue), social functioning, role limitations due to emotional problems, and mental health (psychological distress and psychological well-being). The survey also provides a physical component summary and a mental component summary.

In various aspects, an effective amount of a pulmonary hypertension therapy can improve quality of life of the subject, illustratively as measured by one or more of the health parameters recorded in an SF-36®. survey. For example, an improvement versus baseline is obtained in at least one of the SF-36® physical health related parameters (physical health, role-physical, bodily pain and/or general health) and/or in at least one of the SF-36® mental health related parameters (vitality, social functioning, role-emotional and/or mental health). Such an improvement can take the form of an increase of at least 1, for example at least 2 or at least 3 points, on the scale for any one or more parameters.

### B. Improvement of Prognosis

In another embodiment, the treatment method of the present disclosure can improve the prognosis for a subject having a pulmonary hypertension condition. The treatment of this embodiment can provide (a) a reduction in probability of a clinical worsening event during the treatment period, and/or (b) a reduction from baseline in serum brain natriuretic peptide (BNP) concentration, wherein, at baseline, time from first diagnosis of the condition in the subject is not greater than about 2 years.

Time from first diagnosis, in various aspects, can be, for example, not greater than about 1.5 years, not greater than about 1 year, not greater than about 0.75 year or not greater than about 0.5 year. In one aspect, administration of ranolazine can begin substantially immediately, for example, within about one month or within about one week, upon diagnosis.

In this embodiment, the treatment period is long enough for the stated effect to be produced. Typically, the longer the treatment continues, the greater and more lasting will be the benefits. Illustratively, the treatment period can be at least about one month, for example at least about 3 months, at least about 6 months or at least about 1 year. In some cases, administration can continue for substantially the remainder of the life of the subject.

Clinical worsening event (CWEs) include death, lung transplantation, hospitalization for the pulmonary hypertension condition, atrial septostomy, initiation of additional pulmonary hypertension therapy or an aggregate thereof. Therefore, the treatments of the present disclosure can be effective to provide a reduction of at least about 25%, for example at least about 50%, at least about 75% or at least about 80%, in probability of death, lung transplantation, hospitalization for pulmonary arterial hypertension, atrial septostomy and/or initiation of additional pulmonary hypertension therapy during the treatment period.

Time to clinical worsening of the pulmonary hypertension condition is defined as the time from initiation of a ranolazine treatment regime to the first occurrence of a CWE.

In another particular aspect, the method is effective to provide a reduction from baseline of at least about 15%, for example at least about 25%, at least about 50% or at least about 75%, in BNP concentration.

The pulmonary hypertension condition according to this embodiment can comprise any one or more of the conditions in the WHO or Venice (2003) classification described above. In one aspect, the condition comprises PAH (WHO Group 1), for example idiopathic PAH, familial PAH or PAH associated with another disease.

In various aspects of this embodiment, the subject at baseline exhibits PH (e.g., PAH) of at least WHO Class II, for example Class II, Class III or Class IV as described above.

In a more particular embodiment, the subject at baseline has a resting PAP of at least about 30 mmHg, for example at least about 35 mmHg or at least about 40 mmHg.

### C. Prolongation of Life

In yet another embodiment, the treatment methods of the present disclosure can prolong the life of a subject having a pulmonary hypertension condition, from a time of initiation of treatment, by at least about 30 days. Variants and illustrative modalities of this method are as set forth above.

### D. Extending Time to Clinical Worsening

Still in another embodiment, the present methods can extend time to clinical worsening in a subject having a pulmonary hypertension condition, and decrease the probability of a clinical worsening event by at least about 25%. Variants and illustrative modalities of this method are as set forth above.

### E. Other Treatment Objectives

In any of the methods described hereinabove, the subject can be male or female. For example, the combined drugs can be administered to a female subject according to any of the above methods, including the indicated variants and illustrative modalities thereof. Alternatively, ranolazine can be administered to a male subject, for example a reproductively active male subject, according to any of the above methods, including the indicated variants and illustrative modalities thereof.

In another embodiment, the methods provided herein are useful for treating a pulmonary hypertension condition in a reproductively active male subject, wherein fertility of the subject is not substantially compromised. “Not substantially compromised” in the present context means that spermatogenesis is not substantially reduced by the treatment and that no hormonal changes are induced that are indicative of or associated with reduced spermatogenesis. Male fertility can be assessed directly, for example, by sperm counts from semen samples, or indirectly by changes in hormones such as follicle stimulating hormone (FSH), luteinizing hormone (LH), inhibin B and testosterone.

In one embodiment, a method is provided for treating PAH in a subject, wherein the PAH is associated with one or more of (a) a congenital heart defect, (b) portal hypertension, (c) use of a drug or toxin other than an anorexigen, (d) thyroid disorder, (e) glycogen storage disease, (f) Gaucher disease, (g) hereditary hemorrhagic telangiectasia, (h) hemoglobinopathy, (i) myeloproliferative disorder, (j) splenectomy, (k) pulmonary veno-occlusive disease and/or (l) pulmonary capillary hemangiomatosis. Variants and illustrative modalities of this method are as set forth hereinabove.

Further, in another embodiment, a method is provided for treating a pulmonary hypertension condition classified in WHO Groups 2-5 in a subject. Variants and illustrative modalities of this method are as set forth hereinabove. In one aspect, the condition comprises left-sided atrial or ventricular heart disease and/or left-sided valvular heart disease. In another aspect, the condition is associated with one or more of chronic obstructive pulmonary disease (COPD), interstitial lung disease (ILD), sleep-disordered breathing, an alveolar hypoventilation disorder, chronic exposure to high altitude, a developmental abnormality, thromboembolic obstruction of proximal and/or distal pulmonary arteries, a non-thrombotic pulmonary embolism, sarcoidosis, histiocytosis X, lymphangiomatosis, and/or compression of pulmonary vessels.

As discussed below, ranolazine can be administered in a variety of manners.

### 3. Ranolazine and Methods of Delivery

Methods of the disclosure contemplate a variety of methods of administering ranolazine, including intravenously and orally. In some embodiments, ranolazine is administered in a sustained release formulation. In one embodiment, the aggregate daily dose of ranolazine is about 3000 milligrams, 1500 milligrams, 1000 milligrams, or 750 milligrams. In one embodiment, ranolazine may be administered in a pharmaceutical composition comprising ranolazine and a pharmaceutically acceptable carrier. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic composition is contemplated. Supplementary active ingredients can also be incorporated into the compositions. Methods of administering and formulating ranolazine are well known in the art as described below.

U.S. Pat. No. 4,567,264, discloses ranolazine, and its pharmaceutically acceptable salts, and their use in the treatment of cardiovascular diseases, including arrhythmias, variant and exercise-induced angina, and myocardial infarction. This patent also discloses intravenous (IV) formulations of dihydrochloride ranolazine further comprising propylene glycol, polyethylene glycol 400, Tween 80 and 0.9% saline.

If ranolazine is administered in an IV solution, the solution may comprise a selected concentration of ranolazine of from about Ito about 100 milligrams per milliliter, alternatively about 10 to about 50 milligrams per milliliter or alternatively about 25 milligrams per milliliter. The infusion of the intravenous formulation of ranolazine is initiated such that a target range of ranolazine plasma concentration of about 1000-5000 nanograms base per milliliter (wherein nanograms base per milliliter refers to nanograms of the free base of ranolazine per milliliter) is achieved and sustained.

The presently preferred route of administration for ranolazine and its pharmaceutical acceptable salts and esters is oral. A typical oral dosage form is a compressed tablet, a hard gelatin capsule filled with a powder mix or granulate, or a soft gelatin capsule (softgel) filled with a solution or suspension. U.S. Pat. No. 5,472,707, discloses a high-dose oral formulation employing supercooled liquid ranolazine as a fill solution for a hard gelatin capsule or softgel.

U.S. Pat. No. 6,503,911, discloses sustained release formulations that overcome the problem of affording a satisfactory plasma level of ranolazine while the formulation travels through both an acidic environment in the stomach and a much more basic environment through the intestine, and has proven to be very effective in providing the plasma levels that are necessary for the treatment of angina and other cardiovascular diseases.

In one aspect, a sustained release ranolazine formulation consists essentially of:

| | Weight | Preferred | Most |
|---|---|---|---|
| Ingredient | Range (%) | Range (%) | Preferred (%) |
| Ranolazine | 50-95 | 70-90 | 75 |
| Microcrystalline cellulose (filler) | 1-35 | 5-15 | 10.6 |
| Methacrylic acid copolymer | 1-35 | 5-12.5 | 10.0 |
| Sodium hydroxide | 0.1-1.0 | 0.2-0.6 | 0.4 |
| Hydroxypropyl methylcellulose | 0.5-5.0 | 1-3 | 2.0 |
| Magnesium stearate | 0.5-5.0 | 1-3 | 2.0 |

The sustained release ranolazine formulations can be prepared as follows: ranolazine and pH-dependent binder and any optional excipients are intimately mixed (dry-blended). The dry-blended mixture is then granulated in the presence of an aqueous solution of a strong base which is sprayed into the blended powder. The granulate is dried, screened, mixed with optional lubricants (such as talc or magnesium stearate), and compressed into tablets. Preferred aqueous solutions of strong bases are solutions of alkali metal hydroxides, such as sodium or potassium hydroxide, preferably sodium hydroxide, in water (optionally containing up to 25% of water-miscible solvents such as lower alcohols).

The resulting ranolazine containing tablets may be coated with an optional film-forming agent, for identification, taste-masking purposes and to improve ease of swallowing. The film forming agent will typically be present in an amount ranging from between 2% and 4% of the tablet weight. Suitable film-forming agents are well-known to the art and include hydroxypropyl methylcellulose, cationic methacrylate copolymers (dimethylaminoethyl methacrylate/methyl-butyl methacrylate copolymers-Eudragit® E-Röhm Pharma), and the like. These film-forming agents may optionally contain colorants, plasticizers, and other supplemental ingredients.

U.S. Patent Application Publication 2006/0177502, discloses oral sustained release dosage forms in which the ranolazine is present in 35-50%, alternatively 40-45% ranolazine.

In one embodiment, the ranolazine sustained release formulations include a pH dependent binder, a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example, sodium hydroxide, potassium hydroxide, or ammonium hydroxide, in a quantity sufficient to neutralize the methacrylic acid copolymer to an extent of about 1-20%, for example about 3 to 6%. Suitable pH independent binders include, but are not limited to, hydroxypropylmethylcellulose (HPMC), for example Methocel® E10M Premium CR grade HPMC or Methocel® E4M Premium HPMC. Suitable pharmaceutically acceptable excipients include magnesium stearate and microcrystalline cellulose (Avicel® pH101).

It is further contemplated that for treating pulmonary hypertension, ranolazine can be formulated in 375 mg, 500 mg or 750 mg tablets. Each tablet contains about 75% ranolazine, about 10.0% metllacrylic acid copolymer, about 10.6% microcrystalline cellulose, about 2.0% hydroxypropyl methyl cellulose, about 0.4% sodium hydroxide and about 2.0% magnesium stearate (non bovine).

### 4. Combination Therapies

It is contemplated that ranolazine may be administered in combination with other PAH therapies, including medical therapies and/or supplemental oxygen. Medical therapies recognized in the art to treat PAH include therapeutic agents, such as cardiac glycosides, vasodilators/calcium channel blockers, prostacyclins, anticoagulants, diuretics, endothelin receptor blockers, phosphodiesterase type 5 inhibitors, nitric oxide inhalation, arginine supplementation and combinations thereof.

Combination therapy of ranolazine with a cardiac glycoside is taught in U.S. Patent Application Publication 2010/0130436. Suitable cardiac glycosides include digoxin, oubain, digitoxin, and oleandrin. In one embodiment, the glycoside is digoxin.

Any variety of vasodilators/calcium channel blockers may be used in combination with ranolazine. Examples include, but are not limited to, nifedipine, diltiazem, amlodipine, and combinations thereof.

Further, any variety of prostacyclins may be used in combination with ranolazine. Examples include, but are not limited to, epoprostenol, treprostinil, iloprost, beraprost, and combinations thereof.

Still further, any variety of anticoagulants may be used in combination with ranolazine. Examples include, but are not limited to, warfarin in low doses, phenocoumarol, acenocoumarol (Sintrom®), clorindione, dicumarol, diphenadione, ethyl biscoumacetate, phenprocoumon, phenindione, tioclomarol, and combinations thereof.

Still further yet, any variety of diuretics may be used in combination with ranolazine. Examples include, but are not limited to, aldosterone antagonists.

Endothelin receptor blockers may also be used in combination with ranolazine. Examples include, but are not limited to, bosentan, sitaxsentan, ambrisentan, and combinations thereof. In one embodiment, ranolazine is combined with ambrisentan.

Phosphodiesterase type 5 inhibitors include, but are not limited to, sildenafil citrate, dipyridamole, tadalafil, avanafil, lodenafil, mirodenafil, vardenafil, udenafil and combinations thereof.

In terms of administration, it is contemplated that the two or more agents can be administered simultaneously or sequentially. If the two or more agents are administered simultaneously, they may either be administered as a single dose or as separate doses. Further, it is contemplated that the attending clinician will be able to readily determine the dosage required of the additional agent, the dosing regimen, and the preferred route of administration.

### 5. Additional Formulations

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The ideal forms of the apparatus for administration of the novel combinations for the methods of the disclosure, consist therefore of (1) either a syringe comprising 2 compartments containing the 2 active substances ready for use or (2) a kit containing two syringes ready for use.

In making pharmaceutical compositions that include ranolazine and possibly additional agents, the active ingredients are usually diluted by an excipient and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the disclosure can be formulated so as to provide quick, sustained or delayed release of the active ingredient after administration to the patient by employing procedures known in the art. As discussed above, given the reduced bioavailability of ranolazine, sustained release formulations are generally preferred. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

The compositions are formulated in a unit dosage form. The term “unit dosage forms” refers to physically discrete units suitable as unitary dosages for human subjects and other mammals, each unit containing a predetermined quantity of the active materials calculated to produce the desired therapeutic effect, in association with a suitable pharmaceutical excipient (e.g., a tablet, capsule, ampoule). The active agents of the disclosure are effective over a wide dosage range and are generally administered in a pharmaceutically effective amount. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredients are mixed with a pharmaceutical excipient to form a solid preformulation composition containing a homogeneous mixture of a compound of the present disclosure. When referring to these preformulation compositions as homogeneous, it is meant that the active ingredients are dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. Ranolazine and the co-administered agent(s) can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

### EXAMPLES

The following examples are included to demonstrate embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Ranolazine for Improvement in Right Ventricular (RV) Function in Pulmonary Hypertension

To ascertain the improvement in right ventricular function in patients suffering from pulmonary hypertension or pulmonary arterial hypertension (PAH), the following parameters are examined:
Exercise capacity and quality of life
RV subendocardial perfusion using adenosine cardiac MRI studies
Exercise-induced changes in RV function determined non-invasively using comprehensive Doppler and tissue Doppler echocardiography along with determination of right ventricular—pulmonary vascular coupling and pulmonary vascular impedance spectra
RV contractility and comprehensive pressure-volume analysis using invasive hemodynamics, 2D and 3D echocardiography, and cardiac MRI

Note that the aforementioned endpoints use a combination of invasive and non-invasive techniques. Echocardiography techniques are best suited for exercise studies. Echocardiographic RV volume data can be normalized to MRI RV volume data, and echocardiographic estimation of RV pressures can be normalized to invasive RV pressure data, thereby increasing the accuracy of echocardiographic analysis of RV structure and function.

It is contemplated that when the parameters are examined, results show that ranolazine ameliorates pain associated with pulmonary hypertension or PAH and improve exercise capacity and quality of life in patients with pulmonary hypertension or PAH.

### Example 2

### Beneficial Effects of Ranolazine in a Model of Pulmonary Hypertension and Right-sided Heart Failure

As is discussed in detail hereinabove, ranolazine is an anti-anginal drug that inhibits the late sodium current (I_{Na}) in cardiomyocytes. Late I_{Na} is increased during conditions of ischemia and hypoxia, and causes intracellular Ca²⁺ overload by increasing the amount of intracellular Na⁺ available for exchange via the Na⁺—Ca²⁺ exchanger (NCX). In addition, reactive oxygen species (ROS) have been shown to increase late I_{Na} in ventricular cardiomyocytes, thereby worsening Na⁺ and Ca²⁺ overload. RAN, by inhibiting late I_{Na}, has been shown to improve left-ventricular (LV) function in various models of cardiac ischemia and/or hypoxia where late I_{Na} is increased; however the benefits of RAN on right ventricular (RV) function and pulmonary hemodynamics have not been investigated. Monocrotaline or MCT (a poisonous crystalline alkaloid found in a leguminous plant of the genus Crotalaria) induces progressive pulmonary arterial hypertension (PAH) in rats that results in RV hypertrophy, contractile dysfunction, and eventually RV failure. Both human PAH and MCT-induced PAH models are characterized by hypoxia and increased ROS production but the role of late I_{Na} in this model has not been investigated. Therefore, the present example evaluates the efficacy of RAN to prevent MCT-induced PAH and RV dysfunction.

Male Sprague-Dawley rats were given a single injection of MCT (60 milligrams per kilogram, s.c.) at study inception and randomized into three groups; MCT, low dose RAN (0.25% weight of RAN over weight of chow), and high dose (0.5% weight of RAN over weight of chow) RAN. RAN was given in diet for 28 days mixed in standard rodent chow containing 0.25% or 0.5% RAN for low and high doses, respectively. These concentrations of RAN yielded plasma concentrations of 1-2 micromolar and 5-7 micromolar, respectively. A control group received a sub-cutaneous (s.c.) saline injection and was fed standard rodent chow. Pulmonary hemodynamics, RV function, and RV hypertrophy was assessed at endpoint (day 28 following MCT administration). Alpha-smooth muscle actin (α-SMA) staining on lung tissue sections were carried out to assess pulmonary vascular remodeling at day 28 as well.

MCT caused increases in pulmonary (84±7 vs 29±1 millimeters Hg) and RV (86±7 vs 26±1 millimeters Hg) systolic pressures compared to controls that were dose-dependently reduced by RAN (low dose: 59±5 and 58±5 millimeters Hg) and (high dose: 40±4 and 39±4 millimeters Hg). Both doses of RAN reduced RV hypertrophy [RV(milligrams)/LV(milligrams): 0.4±0.03 and 0.32±0.03 vs 0.55±0.04] and attenuated decreases in RV ejection fraction (−17±11% and −10±8% vs −47±7%) compared to MCT treated animals. MCT also caused an increase in plasma BNP levels (590±106 vs 170±90 picograms/milliliter) that were decreased by both doses of RAN (180±30 and 60±20 picograms/milliliter).

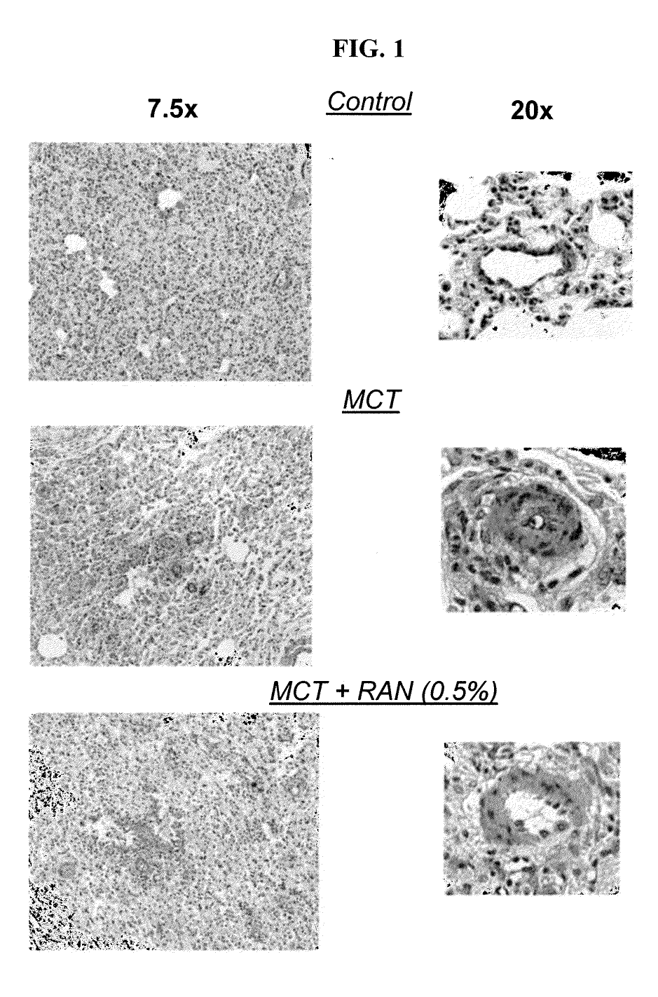

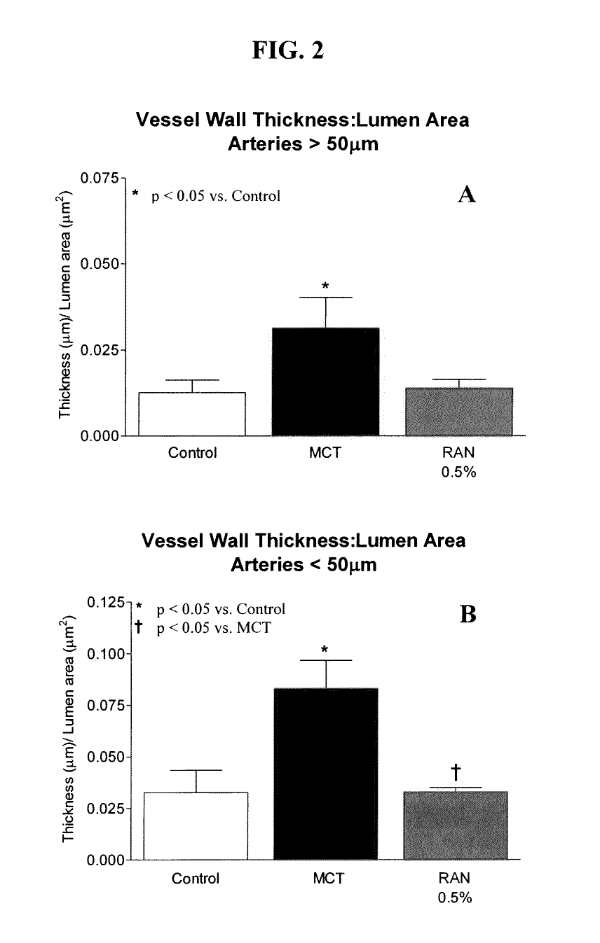

Representative images of α-SMA stained lung sections taken from control animals (control), MCT treated animals (MCT) and animals in the high dose RAN group (MCT+RAN (0.5%) are shown in FIG. 1. Digital quantification of the α-SMA staining was performed for the entire lung sections. The results are shown in FIG. 2A (for lumen area arteries >50 μm) and FIG. 2B (for lumen area arteries <50 μm). As apparent from FIGS. 1, 2A and 2B, MCT administration caused significant increases in pulmonary vascular remodeling as indexed by the ratio of vessel wall thickness to lumen diameter. The pre-acinar pulmonary arteries in RAN-treated animals, however, were similar to those in control animals. Accordingly, RAN significantly reduced remodeling in intra-acinar arteries compared to MCT animals.

Therefore, these data show that RAN significantly and dose-dependently attenuated MCT induced changes in a rodent model of chronic PAH and RV dysfunction.

### Example 3

### Ranolazine Prevents Right Ventricular Remodeling Following Acute Myocardial Infarction in the Mouse

This example demonstrates the effect of ranolazine (RAN) in preventing right ventricular remodeling using a murine model of large anterior wall acute myocardial infarction (AMI) produced by permanent coronary artery ligation of the left coronary artery.

The occurrence of right ventricular (RV) dilatation and dysfunction in AMI and heart failure is an independent negative prognostic factor. Cellular, molecular, and structural changes occur in the RV during AMI, even if the RV is spared from the ischemic insult. Ranolazine is a pharmacologic inhibitor of the cardiac late sodium current and reduces cardiomyocyte calcium overload during ischemia.

Male Imprinting Control Region (ICR) mice underwent permanent coronary artery ligation of the left coronary artery and treated with vehicle (saline) or ranolazine 30 mg/kg i.p. every 6 hours (N=10-12 per group) for 7 days starting at the time of ligation. Infarct size was measured early using Masson's trichrome staining. Transthoracic echocardiography was performed prior to surgery and 7 days later to measure left ventricular and right ventricular dimensions and function.

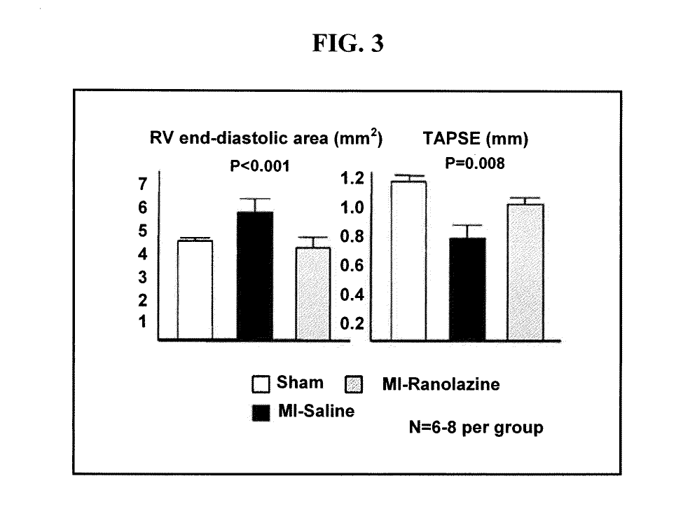

Treatment with ranolazine led to a significant preservation of RV function (measured as tricuspidal annulus plane systolic excursion (TAPSE) and RV fractional area change) and dimension (measured as RV diastolic and systolic areas) in comparison to vehicle-treated mice, in absence of any measurable effects on infarct size or LV function and dimension (FIG. 3).

Therefore, these data show that ranolazine can prevent right ventricular remodeling and dysfunction independent of changes in LV remodeling.

It will be appreciated that those skilled in the art will be able to devise various arrangements which, although not explicitly described or shown herein, embody the principles of the disclosure and are included within its spirit and scope. Furthermore, all conditional language recited herein is principally intended to aid the reader in understanding the principles of the disclosure and the concepts contributed by the inventors to furthering the art, and are to be construed as being without limitation to such specifically recited conditions. Moreover, all statements herein reciting principles, aspects, and embodiments of the disclosure are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

What is claimed is:

1. A method of treating or preventing pulmonary hypertension in a patient in need thereof, said method comprising administering to the patient a therapeutically amount of ranolazine or a salt or salts thereof.

2. The method of claim 1, wherein the pulmonary hypertension is pulmonary arterial hypertension (PAH).

3. The method of claim 2, wherein the pulmonary arterial hypertension is selected from idiopathic PAH, familial PAH, pulmonary veno-occlusive disease (PVOD), pulmonary capillary hemangiomatosis (PCH), persistent pulmonary hypertension of the newborn, or PAH associated with another disease or condition.

4. The method of claim 1, wherein the patient has symptoms comprising chest pain, exertional dyspnea, and/or fatigue.

5. The method of claim 4, wherein the patient has symptoms comprising chest pain.

6. The method of claim 4, wherein the patient has symptoms comprising exertional dyspnea.

7. The method of claim 4, wherein the patient has symptoms comprising fatigue.

8. A method for improving right ventricle function in a patient suffering from pulmonary hypertension, comprising administering to the patient a therapeutically effective amount of ranolazine or a salt or salts thereof.

9. A method for reducing pulmonary arterial pressure in a patient suffering from pulmonary hypertension, comprising administering to the patient a therapeutically effective amount of ranolazine or a salt or salts thereof.

10. A method for treating or ameliorating a symptom in a patient suffering from pulmonary hypertension, comprising administering to the patient a therapeutically effective amount of ranolazine or a salt or salts thereof.

11. The method of claim 10, wherein the symptom comprises chest pain.

12. The method of claim 10, wherein the symptom comprises exertional dyspnea.

13. The method of claim 10, wherein the symptom comprises fatigue.

14. A method of treating or preventing asymptomatic pulmonary hypertension in a patient in need thereof, said method comprising administering to the patient a therapeutically amount of ranolazine or a salt or salts thereof.

15. The method of claim 14, wherein the patient does not suffer from pain or chest pain.

16. The method of any of claim 1, 8-10 or 14, wherein the ranolazine or the salt or salts thereof is administered intravenously.

17. The method of any of claim 1, 8-10 or 14, wherein the ranolazine or the salt or salts thereof is administered orally.

18. The method of any of claim 1, 8-10 or 14, wherein the ranolazine or the salt or salts thereof is administered in a sustained release formulation.

19. The method of any of claim 1, 8-10 or 14, wherein the aggregate daily dose of ranolazine is about 3000 milligrams, 1500 milligrams, 1000 milligrams, 750 milligrams, 500 milligrams or 375 milligrams.

20. A method of treating pulmonary arterial hypertension (PAH) in a patient in need thereof, said method comprising orally administering to the patient a therapeutically amount of ranolazine or a salt or salts thereof, wherein the therapeutically amount contains an aggregate daily dose of ranolazine in the amount of 75 milligrams, 500 milligrams or 375 milligrams.

21. The method of any of claim 1, 8-10, 14 or 20, wherein the patient is also administered an additional therapeutic agent selected from the group consisting of cardiac glycosides, vasodilators/calcium channel blockers, digoxin, an anticoagulant, a diuretic, prostacyclin, an endothelin receptor antagonist, and a phosphodiesterase inhibitor, or a combination thereof.

22. The method of claim 21, wherein the additional therapeutic agent is a vasodilators/calcium channel blockers.

23. The method of claim 21, wherein the additional agent is administered simultaneously with ranolazine or sequentially with ranolazine.

24. The method of claim 23, wherein when the additional agent is administered simultaneously, the agent and ranolazine are administered as a single dose.

25. The method of claim 23, wherein when the additional agent is administered simultaneously, the agent and ranolazine are administered as separate doses.

26. The method of any of claim 1, 8-10, 14 or 20, wherein the patient is further administered oxygen.
